(19) United States
(12) Patent Application Publication
(10) Pub. No.: US 2007/0197978 A1
(43) Pub. Date: Aug. 23, 2007

(54) Drug Delivery Device

(76) Inventor: Leon Wortham, Chattanooga, TN (US)

Correspondence Address: DOUGLAS T. JOHNSON;MILLER & MARTIN, 1000 VOLUNTEER BUILDING, 832 GEORGIA AVENUE, CHATTANOOGA, TN, 37402-2289, US

(21) Appl. No.: 11/676,943

(22) Filed: Feb. 20, 2007

Related U.S. Application Data
(60) Provisional application No. 60/774,568, filed on Feb. 17, 2006.

(51) Int. Cl. A61M 39/08

(52) U.S. Cl. 604/246

(57) ABSTRACT

A portable drug delivery device is provided for sustained administration of selected medications from a plurality of medications stored in internal of fluid cassettes within the device.

### RELATED U.S. APPLICATION DATA

The present application claims priority to U.S. Provisional Application No. 60/774,568, filed on Feb. 17, 2006 which is incorporated herein by reference.

### FIELD OF THE INVENTION

The present invention relates to drug delivery devices, and particularly to portable devices designed to provide sustained drug delivery.

### BACKGROUND OF THE INVENTION

Despite innovations in modern medicine and technology, the modern battlefield is more lethal today than ever before. Soldiers have to be prepared for a multitude of different environments that may change almost without notice and may delay the provision of comprehensive medical care to wounded or injured soldiers for hours. Similarly, injuries sustained by persons in remote areas may not be subject to comprehensive treatment for a number of hours, and therefore there is a need for a sustained drug delivery system that can be self administered or applied by a companion with minimal training on the battlefield or in a wilderness or other remote location.

### SUMMARY OF THE INVENTION

The device is designed to treat immediate needs and to stabilize an injured person or victim by providing sustained medical treatment to the victim for an extended period of time until the victim can receive the needed comprehensive medical attention. The device may be manufactured in a compact form only about 2 inches by 3 inches in size, about the size of a deck of cards or a pack of cigarettes, and strapped, as by hook and loop fastener material, to the arm or leg. The device is designed to inject and deliver three drugs, mostly typically morphine, erythropoietin, and an antibiotic. The dosage administered is based on vital signs taken directly from the injured victim. An onboard processor provides instructions to dispense and dose monitor the victim and also to allow this information to be uploaded and recorded once the victim has been returned to a medical station or hospital for comprehensive treatment.

### BRIEF DESCRIPTION OF THE DRAWINGS

The invention may be more fully appreciated with reference to the following drawings in which:

FIG. 1 is a schematic view of an interior of a drug delivery device according to the invention.

FIGS. 2A-2C are sectional views of piston designs corresponding to three different drug modules.

FIGS. 3A-3C are top plan views of diagrams for fluid cassettes to be utilized with corresponding pistons of FIGS. 2A-2C.

FIG. 4 is a top plan view of a device according to the present invention showing a user interface.

FIG. 5 is an exemplary representation of interior circuit boards for holding embedded instructions and a processor to control the delivery device.

### DETAILED DESCRIPTION OF THE INVENTION

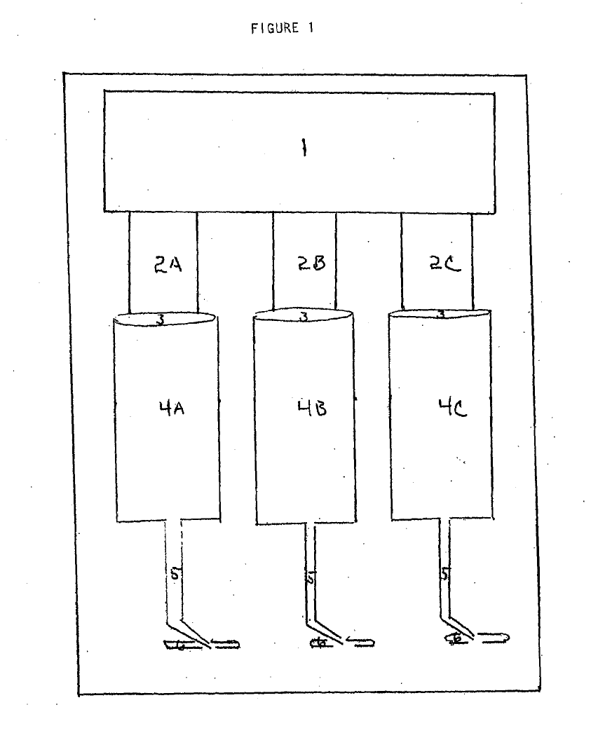

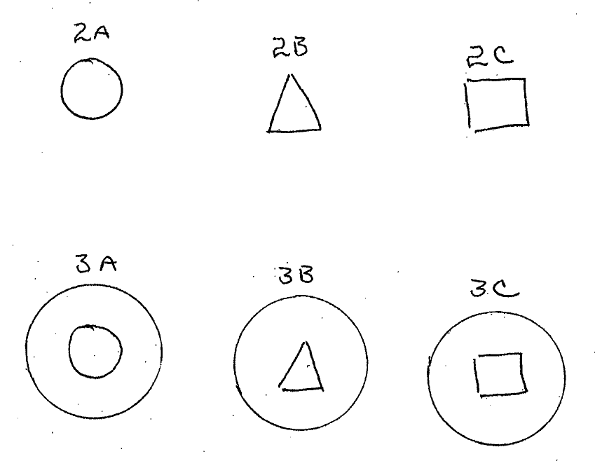

Fluid Cassette & Injection Needle Design: FIG. 1 illustrates a schematic of a exemplary structure for carrying and containing the injectable fluids. Considerations for leakage, pressurization, ruggedization, loading ease, reliable fluid removal, disposal and sterility are addressed with options including self-contained insertable cylinders, collapsible fluid bags and fluid zones molded as part of cassettes 4A-4C. The needles 5 design incorporates needle length, gage, flexure, connection to fluid vessel and engagement method (manual, automatic or both). Other aspects permit stand alone hand actuation of an individual cassette injector, without use of the power unit, such as motor drive unit 1. For manual use the cassettes incorporate a visual scale for the amount to be injected.

Drive System Selection & Dose Monitoring: The selection of the fluid reservoir type somewhat determines the appropriate method for precision fluid metering. Alternative designs include motor drive systems 1 or pressurized vessels with a CO2 power cartridge. The most typical power source utilizes electrically powered brushed DC, brushless DC, servo, stepper or hollow nut motors operating an indexing device to move a piston 2A-2C. The most suitable indexing devices are selected from: ball screw, rigid rapier, slotted belt, flexible rapier, lever camming, rack and pinion, and worm gear. Closed loop feedback, using magnetic scales or optical encoders, of the motor or indexing means enable precise dose dispensing to be achieved. Flow monitoring and feedback are other possible means of closed loop control of the fluid stream. The processor 10 is instructed by embedded software within chipset 11 to perform dispensing calculations based on body weight and medicine type.

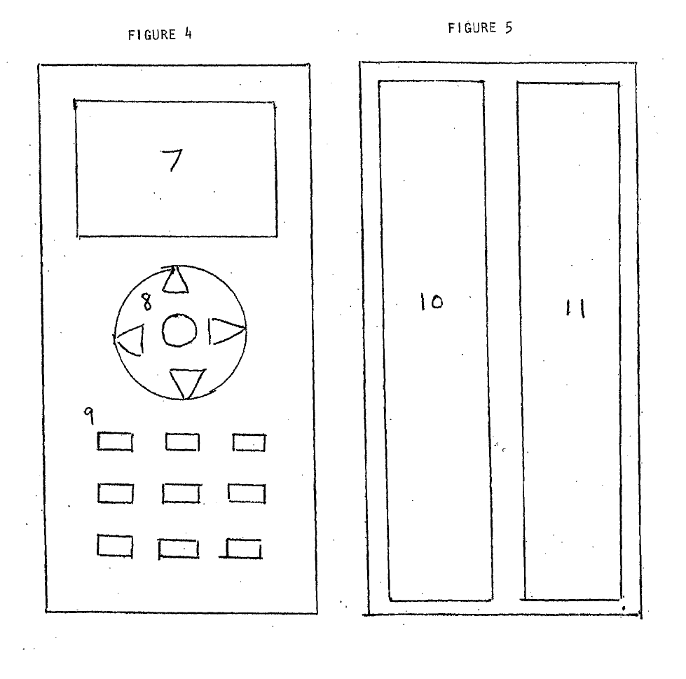

User Interface, Display & Buttons: FIG. 4 illustrates the human interface logic required to retain ergonomic control of the dispensing unit. User interface consists of LCD display 7 and membrane pushbutton keypad 9 and scroll pad 8 for easy operation of the drug delivery device. The specific type of membrane pushbuttons may include metal cap, hall effect and carbon coated bridging of circuit board traces. Backlighting of the LCD display is provided and the means for backlighting can include electro-luminescent, LED, CCFL or defused light tubes. A cuttable jumper can permanently disable backlighting for special situations.

Cassette Communication & Coding: Several possible methods or means for identifying cassettes 4A-4C exist: movable tabs as on a floppy disk, DIP switches, blocked or open optical casement holes, breakable VCR-like tabs, gold contacts like on 35 mm film cartridges or radio frequency excited identification (RFID). Another identification avenue includes flash or EEPROM memory chips with four wire connections. A simple method of identifying individual cassettes is to use breakable plastic tabs, much like that on a VCR tape. Safety confirmation logic may require all three cassettes 4A-4C to retain different IDs so that individual bays look for a specific ID/medication type. Any two IDs alike or a wrong ID in a specific bay may be programmed to disable injection. Color coding and shape keying of individual cassettes and bays provide an additional layer of safety. Once the medication type is known, the dose and frequency is administered from preprogrammed recipe retained by the microprocessor 10 and chipset 11.

Data Downloading & Memory: Data storage memory is provided with the dispenser and a means of downloading or inputting body weight, dose amount, time and frequency for each drug. Data communication protocols can include: IRDA, RFID, USB 2.0, Firewire IEEE 1394, RS-232 or RS-484. The device is preferably battery powered and has memory back-up and also incorporates a means for bi-directional communication enabling software changes and updates to the microprocessor.

Software Integration & Microprocessor Programming: The predetermined logic to operate the device is preferably embedded into an appropriate memory microprocessor package. The microprocessor 10 retains all logic needed to operate the drug delivery device.

PCB Design: A printed circuit board design shrinks all bread-boarded components into a small package. Design options include: single versus double sided, surface mount versus through hole and epoxy laminate versus mylar thin film and incorporate connection to the LCD display, connection to the battery, connection to the cassettes for identification and mounting method for securing the PCB and LCD display.

Environmental, Ruggedization, Sterility, Packaging & Case Design: Manufacture of the device should place all components inside a case that effects a protective envelope. The case design reflects environmental and ruggedization aspects, such as contamination, water resistance, UV resistance, impact resistance, abrasion resistance, vibration dampening and sterility. Additional features include:

Cassette Encoding Module: A stand alone module for automatically encoding the cassette with the appropriate drug identification, including color coded labeling and programming of internal cassette chip sets or tab punching. If keyed and color coded cassettes are used this device automatically selects the matched preloaded cassette, from a stacked magazine, to the appropriate drug to be filled.

Data Review Software: Software is provided to enable an external monitor or parent device such as a PDA, laptop or desktop PC to communicate with the drug delivery device. This is the actual software loaded on the PDA or PC, not the injection device, and provides features including data-logging of drug type, dose, frequency, date, time and body weight in a tabulation or graphical format.

Cassette Filling Station: The cassette filling module enables cassettes to be preloaded into a stacked magazine for automatic filling of the appropriate drug matched to the cassette in a hermetically sealed package.

Although preferred embodiments of the present invention have been disclosed in detail herein, it will be understood that various substitutions and modifications may be made to the disclosed embodiment described herein without departing from the scope and spirit of the present invention as recited in the appended claims.

1. A portable drug delivery device for sustained administration of a plurality of drugs comprising:
a power source;
a drive system;
a plurality of fluid cassettes, each cassette containing a different medication;
an indexed piston in communication with the drive system for each fluid cassette;
a processor for controlling the motor drive system;
a feed back loop to apprise the processor of the dose of medication dispensed from each fluid cassette;
a needle connected to each fluid cassette for injecting medication into a patient;
a user interface comprising a display and data entry means;
wherein each fluid cassette is uniquely identified according to the type of medicine contained for placement in the device.

2. The drug delivery device of claim 1 further comprising a communication port to electronically communicate dispensing history to a monitoring device.

3. The drug delivery device of claim 1 wherein the device monitors at least one vital sign of the patient.

4. The drug delivery device of claim 1 further comprising straps to secure the device about an extremity of a patient.

5. Drug delivery device of claim 1 further comprising a UV and water resistant casing enclosing the drive system and fluid cassettes.
